(19) United States
(12) Patent Application Publication
(10) Pub. No.: US 2007/0185562 A1
(43) Pub. Date: Aug. 9, 2007

(54) Medical device for unstable and vulnerable plaque

(75) Inventor: Joseph G. Furst, Lyndhurst, OH (US)

Correspondence Address: FAY SHARPE LLP, 1100 SUPERIOR AVENUE, SEVENTH FLOOR, CLEVELAND, OH, 44114, US

(73) Assignee: JGF Company

(21) Appl. No.: 11/703,315

(22) Filed: Feb. 7, 2007

Related U.S. Application Data
(60) Provisional application No. 60/771,508, filed on Feb. 8, 2006.

(51) Int. Cl. A61F 2/06

(52) U.S. Cl. 623/1.15; 606/195; 623/1.42

(57) ABSTRACT

A medical device used to treat unstable and/or vulnerable plaque in a blood vessel so as to minimize or prevent rupture of the unstable and/or vulnerable plaque at least until the unstable and/or vulnerable plaque is at least partially encapsulated by the medical device.

The present invention relates generally to medical devices, more particularly to a medical device that can be inserted into a blood vessel to improve the blood flow through the blood vessel, and even more particularly to a stent that is inserted into a diseased blood vessel to heal, repair and/or encapsulate unstable and/or vulnerable plaque in the blood vessel.

### BACKGROUND

Vulnerable plaque is a type of fatty buildup in a blood vessel (e.g., artery, etc.) thought to be caused by inflammation. The plaque is covered by a thin, fibrous cap that upon rupture may lead to the formation of a blood clot and, ultimately, occlusion of the blood vessel. Plaque rupture most often occurs in smaller blood vessels such as, but not limited to, the coronary arteries, which supply blood to the heart muscle. The occlusion of a coronary artery can lead to a heart attack. Rupture of the plaque can also or alternatively result in damage to the blood vessel thereby potentially resulting in restenosis.

Medical devices such as, but not limited to stents, angioplasty balloons, etc., are commonly used to improve the flow of blood through a blood vessel. A blood vessel that includes plaque can have obstructed blood flow through the area of plaque. This obstructed blood flood can cause damage to the blood vessel, damage to one or more organs being supplied blood by the blood vessel, unacceptably increase blood pressure in one or more regions of the body, etc. The medical devices use to treat regions that include vulnerable plaque are typically designed to compress or flatten the plaque against the wall of the blood vessel so as to improve the flow of blood through the blood vessel. Due to the relatively fragile nature of some plaque and/or the size of the plaque, the compression or flattening of the plaque by such medical devices can cause the plaque to rupture and/or be damaged, which in turn can result in undesirable consequences.

In view of the current state of technology with regard to medical devices for use in a blood vessel, there is a need for a medical device that can improve the blood flow through a blood vessel that is impaired by the existence of plaque and which medical device can at least partially encapsulate the plaque so as to inhibit or prevent portions of the plaque from causing or resulting in an undesired medical event after the medical device has been expanded in the region of the plaque.

### BRIEF SUMMARY

The present invention is directed to a medical device such as, but not limited to a stent, that is used improve the blood flow through a blood vessel that is impaired by the existence of plaque and which medical device inhibits or prevents ruptured plaque from traveling downstream in the blood vessel in the event that the unstable and/or vulnerable plaque ruptures during the use of the medical device. The present invention is also directed to a method for using the medical device.

In one non-limiting aspect of the invention, the medical device is designed to partially or fully encapsulate a diseased area in a body passageway so as to inhibit or prevent one or more portions of the diseased area from escaping the diseased area once the medical device has been at least partially inserted in the region of the diseased area. As used herein, the term “body passageway” is defined to be any passageway or cavity in a living organism (e.g., bile duct, bronchial tube, nasal cavity, blood vessel, heart, esophagus, trachea, stomach, fallopian tube, uterus, ureter, urethra, the intestines, lymphatic vessel, nasal passageway, eustachian tube, acoustic meatus, etc.). For vascular applications, the term “body passageway” primarily refers to blood vessels and the heart. In one non-limiting embodiment of the invention, the medical device is designed to inhibit or prevent one or more portions of a diseased and/or damaged area (e.g., tissue tear, cancer, vulnerable plaque, unstable plaque, plaque, infection, blood clot, etc.) in a vascular organ (e.g., heart, blood vessel, etc.) that has become dislodged and/or has ruptured in the vascular organ during and/or after the placement of the medical device at the treatment area from traveling downstream from the diseased area. For instance, when the body passageway is a blood vessel and the diseased area includes unstable and/or vulnerable plaque, the medical device is designed to partially or fully cover and/or encapsulate the unstable and/or vulnerable plaque so that if the unstable and/or vulnerable plaque ruptures, the ruptured portion of the unstable and/or vulnerable plaque is inhibited or prevented by the medical device from escaping the diseased area, and thereby inhibiting or preventing a clot and/or other undesirable medical incidence from occurring.

In another and/or alternative non-limiting aspect of the invention, the medical device is in the form of a stent. The stent can be at least partially formed of one or more polymers, metals (e.g., aluminum, barium, bismuth, calcium, carbon, cobalt, copper, chromium, depleted radioactive elements, gold, iron, lead, molybdenum, magnesium, nickel, niobium, platinum, rare earth metals, rhenium, silver, tantalum, titanium, tungsten, vanadium, yttrium, zinc, zirconium, and/or alloys thereof), ceramics, and/or fiber reinforced materials (e.g., carbon fiber material, fiberglass, etc.). The one or more materials that are selected to form one or more portions of the stent are generally selected to impart the desired properties on the stent so that the stent can 1) withstand the manufacturing process that is needed to produce the medical device (e.g., laser cutting, etching, MEMS (e.g., micro-machining, etc.) processes, masking processes, crimping, annealing, drawing, pilgering, electroplating, electro-polishing, chemical polishing, ion beam deposition or implantation, sputter coating, vacuum deposition, molding, melting, adhesive bonding, cutting, extruding, etching, heating, cooling, etc.); and/or 2) impart the desired properties to the medical device (e.g., strength, durability, biostability, biodegradability, bendability, radial strength, flexibility, tensile strength, biocompatibility, etc.). In one non-limiting embodiment of the invention, the stent includes one or more body sections. In another and/or alternative non-limiting embodiment of the invention, at least one body section of the medical device includes first and second ends and a wall surface disposed between the first and second ends.

In still another and/or alternative non-limiting embodiment of the invention, the medical device has a first cross-sectional area which permits delivery of the body section into the blood vessel, and a second, expanded cross-sectional area. The second, expanded cross-sectional area has a size that typically enables the expanded portion of the stent to be secured or anchored to the inner wall of the body passageway; however, this is not required. The expansion of the medical device typically occurs by a radially, outwardly extending force applied at least partially from the interior region of the body section (e.g., use of a balloon, balloon catheter, etc.), and/or by use of a memory material (e.g., Nitinol, memory polymer, etc.). The second, expanded cross-sectional area of one or more portions of the one or more body sections can be variable; however, this is not required. When one or more portions of the one or more body sections have a variable second cross-sectional area, the variable cross sectional area typically dependents upon the amount of radially outward force applied to the one or more body sections; however, this is not required. When the medical device includes two or more body sections, the two or more body sections can be connected together by at least one connector. The one or more connectors, when use, can be designed to allow for flexible movement through the body passageway; however, this is not required. The medical device can include rounded, smooth and/or blunt surfaces on one or more regions of the medical device so as to minimize and/or prevent damage to the body passageway as the medical device is inserted in and/or through the body passageway and/or expanded in the body passageway; however, this is not required.

in still another and/or alternative non-limiting aspect of the invention, the medical device includes one or more of the body sections that have a thin wall thickness in one or more regions of the medical device. In one non-limiting embodiment of the invention, the wall thickness on one or more portions of the one or more body sections is less than about 0.2 inch. In one non-limiting aspect of this embodiment, the wall thickness on one or more portions of the one or more body sections is less than about 0.15 inch. In another non-limiting aspect of this embodiment, the wall thickness on one or more portions of the one or more body sections is less than about 0.1 inch. In still another non-limiting aspect of this embodiment, the wall thickness on one or more portions of the one or more body sections is about 0.005-0.1 inch. In yet another non-limiting aspect of this embodiment, a majority of one or more body sections of the medical device has an average wall thickness of about 0.005-0.2 inch. In still yet another non-limiting aspect of this embodiment, a majority of one or more body sections of the medical device has an average wall thickness of about 0.001-0.05 inch. In still another non-limiting aspect of this embodiment, a majority of one or more body sections of the medical device has an average wall thickness of about 0.01-0.04 inch. In yet another non-limiting aspect of this embodiment, a majority of one or more body sections of the medical device has an average wall thickness of about 0.01-0.032 inch.

In yet another and/or alternative non-limiting aspect of the invention, one or more body sections of the medical device can be expanded by the use of a small internal force or pressure. In one non-limiting aspect of this embodiment, an inflation device (e.g., balloon, balloon catheter, etc.) is use to expand one or more portions of the body of the medical device when the medical device is positioned in a treatment area in the blood vessel. In another and/or alternative non-limiting aspect of this embodiment, low pressures can be applied to the inflation device so as to slowly inflate the inflation device which in turn causes one or more portions of the one or more body sections of the medical device to slowly expand in a treatment area of a body passageway. In one non-limiting particular design of the medical device, the average rate of expansion of one or more portions of the one or more body sections of the medical device in the body passageway is less than about 60 inches per minute. In another non-limiting particular design of the medical device, the average rate of expansion of one or more portions of the one or more body sections of the medical device in the body passageway is less than about 20 inches per minute. In still another non-limiting particular design of the medical device, the average rate of expansion of one or more portions of the one or more body sections of the medical device in the body passageway is less than about 5 inches per minute. In yet another non-limiting particular design of the medical device, the average rate of expansion of one or more portions of the one or more body sections of the medical device in the body passageway is less than about 1 inch per minute. In still yet another non-limiting particular design of the medical device, the maximum pressure applied to the inflation device to at least partially inflate the inflation device and to cause one or more portions of the one or more body sections of the medical device in the body passageway to expand is less than 60 psi. In a further non-limiting particular design of the medical device, the maximum pressure applied to the inflation device to at least partially inflate the inflation device and to cause one or more portions of the one or more body sections of the medical device in the body passageway to expand is less than 20 psi. In still a further non-limiting particular design of the medical device, the maximum pressure applied to the inflation device to at least partially inflate the inflation device and to cause one or more portions of the one or more body sections of the medical device in the body passageway to expand is less than 10 psi. As can be appreciated other maximum pressures can be used to at least partially inflate the inflation device and to cause one or more portions of the one or more body sections of the medical device in the body passageway to expand.

In still yet another and/or alternative non-limiting aspect of the invention, the medical device is designed so that one or more portions of the medical can be expanded to at least partially secure the medical device in a body passageway while one or more other sections of the medical device are not ever further expanded or not initially further expanded. The variable expansion of the medical device enables the medical device to be expanded in regions of the body passageway that are fully or partially spaced from the diseased region of the body passageway. In one non-limiting embodiment of the invention, one or more portions of the medical device are designed to be initially expanded so as to at least partially secure or anchor the medical device in position in or about a diseased area of a body passageway. In one non-limiting aspect of this embodiment, the medical device is configured and sized so that one or more end portions of the medical device can be initially expanded in regions that are spaced from the diseased area so to not damage, injure and/or rupture the diseased area when the medical device is at least partially secured in the body passageway. In another and/or alternative non-limiting aspect of this embodiment, the medical device includes a body section having a longitudinal axis and wherein the body section is designed to be initially expanded at both end portions while not expanding or limiting the expansion of the body section between the end portions so as to form a body section having a generally dog-bone shape. As can be appreciated, the body section can be initially expanded to have many other configurations.

In another and/or alternative non-limiting embodiment of the invention, the body section of the medical device has a longitudinal length that sufficient to fully transverse the longitudinal length of the diseased area in the body passageway. In one non-limiting aspect of this embodiment, the body section of the medical device has a longitudinal length to enable one or more end portions of the body section to be initially expanded so as to at least partially secure or anchor the body section to the body passageway at a location that is spaced from the diseased area in the body passageway and which unexpanded or less expanded portion of the body section fully transverses the longitudinal length of the diseased area in the body passageway. In another and/or alternative non-limiting aspect of this embodiment, the body section of the medical device has a longitudinal length to enable one or more end portions of the body section to be initially expanded so as to at least partially secure or anchor the body section to the body passageway at a location that is spaced about 0.001-5 mm from the diseased area in the body passageway and which unexpanded or less expanded portion of the body section fully transverses the longitudinal length of the diseased area in the body passageway. In still another and/or alternative non-limiting aspect of this embodiment, the body section of the medical device has a longitudinal length to enable one or more end portions of the body section having a length of about 1-10 mm to be initially expanded so as to at least partially secure or anchor the body section to the body passageway at a location that is spaced from the diseased area in the body passageway and which unexpanded or less expanded portion of the body section fully transverses the longitudinal length of the diseased area in the body passageway. In yet another and/or alternative non-limiting aspect of this embodiment, the body section of the medical device has a longitudinal length to enable one or more end portions of the body section having a length of about 2-5 mm to be initially expanded so as to at least partially secure or anchor the body section to the body passageway at a location that is spaced from the diseased area in the body passageway and which unexpanded or less expanded portion of the body section fully transverses the longitudinal length of the diseased area in the body passageway. In still yet another and/or alternative non-limiting aspect of this embodiment, the body section of the medical device has a longitudinal length to enable one or more end portions of the body section having a length of about 1-10 mm to be initially expanded so as to at least partially secure or anchor the body section to the body passageway at a location that is spaced about 0.001-5 mm from the diseased area in the body passageway and which unexpanded or less expanded portion of the body section fully transverses the longitudinal length of the diseased area in the body passageway.

In a further and/or alternative non-limiting aspect of the invention, the medical device includes a flexible barrier that is designed to inhibit or prevent the penetration of fragments and/or other substances from a diseased area from penetrating the flexible barrier. The flexible barrier can be designed to be partially or fully secured to one or more portions of the medical device (e.g., secured to the complete body section, secured only at the end portions of body section, secured to one or more outer surfaces of the body section, secured to one or more inner surfaces of the body section, etc.) by a variety of mechanisms (e.g., coating one or more portions of the body section, adhesively connected to one or more portions of the body section, clamped to one or more portions of the body section, melt bonded to one or more portions of the body section, stretch fitted to one or more portions of the body section, etc.). In one non-limiting embodiment of the invention, the flexible barrier is directly connected to one or more portions of the medical device by one or more mechanisms (e.g., coating one or more portions of the body section, adhesively connected to one or more portions of the body section, clamped to one or more portions of the body section, melt bonded to one or more portions of the body section, etc.). In another and/or alternative non-limiting embodiment of the invention, the flexible barrier is at least partially indirectly connected to one or more portions of the medical device. In one non-limiting aspect of this embodiment, the flexible barrier is at least partially in the form of a sleeve or sheath that is inserted about at least a portion of the outer peripheral surface of the medical device in a loose or snug manner and which secures to the one or more portions of the medical device when one or more portions of the medical device are expanded thereby causing the sleeve to be stretched into contact with and secured to one or more portions of the medical device. As can be appreciated, many other or additional configurations of the flexible barrier can be used to directly and/or indirectly secure the flexible barrier to the medical device. In another and/or alternative non-limiting embodiment of the invention, the flexible barrier includes a stretchable material to enable the flexible barrier to expand without tearing when one or more portions of the medical device are expanded. In one non-limiting aspect of this embodiment, the flexible barrier includes a flexible and a stretchable material. The flexible and stretchable material can be formed of one or more materials.

In still another and/or alternative non-limiting embodiment of the invention, the flexible barrier can be applied directly onto one or more portions of the medical device by a variety of processes (e.g., spraying [atomizing spray techniques, etc.], dip coating, roll coating, sonication, brushing, plasma deposition, depositing by vapor deposition, etc.); or can be at least partially preformed (e.g., formed into a tubular sleeve, etc.) and subsequently inserted on and/or connected to the medical device. In still another and/or alternative non-limiting embodiment of the invention, the flexible barrier is at least partially formed of one or more polymers. The one or more polymers can be biostable, biodegradable, or bioabsorbable. Non-limiting examples of polymers that are considered to be biodegradable, bioresorbable, or bioerodable and which can be used to form one or more portions of the flexible barrier and/or one or more other portions of the medical device include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g., DL-PLA), with and without additives (e.g., calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above.

Non-limiting examples of polymers that considered to be biostable and which can be used to form one or more portions of the flexible barrier and/or one or more other portions of the medical device include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®)); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; keratin; chitin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g., polystyrene); poly(vinyl ethers) (e.g., polyvinyl methyl ether); poly(vinyl ketones); poly(vinylidene halides) (e.g., polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g., polyvinyl chloride); polyacrylic acid; ethylene acrylic acid copolymer; ethylene vinyl acetate copolymer; polyvinyl alcohol; poly(hydroxyl alkylmethacrylate); Polyvinyl esters (e.g., polyvinyl acetate); and/or copolymers, blends, and/or composites of above. Non-limiting examples of polymers that can be made to be biodegradable and/or bioresorbable with modification and which can be used to form one or more portions of the flexible barrier and/or one or more other portions of the medical device include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. In non-limiting embodiment of the invention, the flexible barrier is at least partially preformed from a sleeve or sheath of material. Such sheath or sleeve can have a generally circular cross-sectional shape; however, other shapes can be formed. In one non-limiting aspect of this embodiment, the sleeve or sheath of flexible barrier can be at least partially formed from an amphiphilic block copolymer. Non-limiting examples of such polymers are disclosed in US 2005/0165476 published on Jul. 28, 2005 and entitled “Vascular Grafts With Amphiphilic Block Copolymer Coatings”, which is incorporated herein by reference.

In still a further and/or alternative non-limiting aspect of the invention, the medical device includes a flexible barrier that is designed to at least partially encapsulate a diseased portion of a body passageway between the wall of the body passageway and the flexible barrier when the medical device is at least partially expanded. In one non-limiting embodiment of the invention, the medical device is designed and sized so that at least two end portions of the medical device are expandable to enable the end portions to secure the medical device to the inner wall of the body passageway at a location spaced from the distal and proximal ends of the diseased area in the body passageway. The expansion of the medical device results in at least a portion of the flexible barrier to engage or closely engage the inner wall of the body passageway at location spaced from the distal and proximal ends of the diseased area in the body passageway in a manner to inhibit or prevent penetration of fragments and/or other substances from a diseased area from penetrating the flexible barrier at the expanded end portions of the medical device. The remainder of the flexible barrier completes the encapsulation of the diseased area between the flexible barrier and the inner wall of the body passageway. A portion of the medical device between the end portions of the medical device is designed to not be expanded or expanded as much as the end portions until after the end portions have been sufficiently expanded in the body passageway to at least partially anchor or secure the medical device in the body passageway. By not expanding or by not initially expanding the intermediate portion of the medical device by the same amount as the expanded end portions, the medical device minimizes or does not cause any pressure to be exerted on the diseased area during the initial expansion and anchoring of the medical device in the body passageway; thereby resulting in a reduced incidence of damage, injury or rupture of the diseased area until after the at least two end portions of the medical device have been sufficiently expanded in the body passageway to at least partially secure or anchor the medical device in the body passageway and to caused the diseased area to be encapsulated between the flexible barrier and the inner wall of the body passageway. Once the diseased area has been encapsulated by use of the medical device, the intermediate portion of the medical device can be designed to be expanded or further expanded; however, this is not required. The expansion of the intermediate portion of the medical device can be used to improve fluid flow through the body passageway. When the intermediate portion is expanded or further expanded, the diseased area can be damaged, injured and/or ruptured (e.g., rupture of vulnerable plaque, rupture of unstable plaque, etc.); however, if such damage, injury and/or rupture to the diseased area does occur, the flexible barrier inhibits or prevents the cell, fluids, particles, etc. from the diseased area from escaping the encapsulated area.

In yet a further and/or alternative non-limiting aspect of the invention, an inflation device is used to expand one or more end portions of the medical device so as to at least partially secure the medical device in the body passageway. In one non-limiting embodiment, one or more inflation devices (e.g., balloon, balloon catheter, etc.) are designed to expand at least two end portions of the medical device and to then cause an intermediate portion that is portioned between two end portions to not be expanded or be expanded an amount that is less than the end portions. In one non-limiting aspect of this embodiment, the same inflation device or a different inflation device can be designed to further expand the intermediate portion after the at least two end portions of the medical device have been expanded, if such expansion of the intermediate portion is desired. In one non-limiting design in accordance with this aspect, the same inflation device is used to further expand the intermediate portion after (e.g., few second, few minutes, few hours, few days, few months, etc.) the at least two end portions of the medical device have been expanded. The inflation device can include multiple chambers and/or inflation arrangements to enable one portion of the inflation device to be expanded differently at a different time from one or more other portions of the inflation device. In another non-limiting design in accordance with this aspect, one inflation device is used to expand the at least two end portions of the medical device and another inflation device is used to further expand the intermediate portion after (e.g., few second, few minutes, few hours, few days, few months, etc.) the at least two end portions of the medical device have been expanded. In still another non-limiting design in accordance with this aspect, the same inflation device is used to expand the at least two end portions of the medical device and to further expand the intermediate portion after the at least two end portions of the medical device have been expanded. The inflation device is initially used to expand the at least two end portions of the medical device. After the at least two end portions are expanded, the first inflation device is repositioned in the medical device and expanded so as to cause at least a portion of the intermediated portion to become more expanded.

In still yet a further and/or alternative non-limiting aspect of the invention, the medical device can be at least partially anchored in the vessel wall of a blood vessel.

In another and/or alternative non-limiting aspect of the invention, the medical device can deliver one or more biological agents locally and/or regionally at or about a treatment area (e.g., diseased area, region about a diseased area, etc.). The one or more biological agents can be at least partially formulated to increases the healing response of the treated area. The one or more biological agent can be contained, included and/or coated on one or more regions of the flexible barrier and/or one or more regions of the medical device other than the flexible barrier.

The term “biological agent” includes, but is not limited to, a substance, drug or otherwise formulated and/or designed to prevent, inhibit and/or treat one or more biological problems, and/or to promote the healing in a treated area. Non-limiting examples of biological problems that can be addressed by one or more biological agents include, but are not limited to, viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; promote healing of unstable and/or vulnerable plaque; and/or the like. Non-limiting examples of biological agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; agents that modulate intracellular Ca²⁺ transport such as L-type (e.g., diltiazem, nifedipine, verapamil, etc.) or T-type Ca²⁺ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; anti-fungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca₂₊/calmodulin kinase II, casein kinase II, etc.); aspirin and/or derivatives thereof; azathioprine and/or derivatives thereof; -Estradiol and/or derivatives thereof; -1-anticollagenase and/or derivatives thereof; calcium channel blockers and/or derivatives thereof; calmodulin antagonists and/or derivatives thereof (e.g., H₇, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof; endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP₃ receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-1 and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H₃P³²O₄, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; stem cells; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; T_{H1} and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof.

As can be appreciated, the biological agent can include one or more derivatives of the above listed compounds and/or other compounds. The type and/or amount of biological agent contained, included in and/or coated on the medical device can vary. When two or more biological agents are contained, included in and/or coated on the medical device, the amount of two or more biological agents can be the same or different. The type and/or amount of biological agent contained, included on, part of and/or in the medical device is generally selected for the treatment of the diseased area in a body passageway; however, the one or more biological agents can be selected for other or additional uses. The one or more biological agents can be coated on and/or impregnated in the medical device by a variety of mechanisms such as, but not limited to, spraying (e.g., atomizing spray techniques, etc.), dip coating, roll coating, sonication, brushing, plasma deposition, depositing by vapor deposition.

In still another and/or alternative non-limiting aspect of the invention, the one or more biological agents on, part of and/or in the medical device, when used on the medical device, can be released in a controlled manner. As can be appreciated, controlled release of one or more biological agents on the medical device is not always required and/or desirable. As such, one or more of the biological agents on, part of and/or in the medical device can be uncontrollably released from the medical device during and/or after insertion of the medical device in the treatment area. It can also or alternatively be appreciated that one or more biological agents on, part of and/or in the medical device can be controllably released from the medical device and one or more biological agents on, part of and/or in the medical device can be uncontrollably released from the medical device. It can also or alternatively be appreciated that one or more biological agents on, part of and/or in one region of the medical device can be controllably released from the medical device and one or more biological agents on, part of and/or in the medical device can be uncontrollably released from another region on the medical device. As such, the medical device can be designed such that 1) all the biological agent on, part of and/or in the medical device is controllably released, 2) some of the biological agent on, part of and/or in the medical device is controllably released and some of the biological agent on the medical device is non-controllably released, or 3) none of the biological agent on, part of and/or in the medical device is controllably released. The medical device can also or alternatively be designed such that the rate of release of the one or more biological agents from the medical device is the same or different. The medical device can also or alternatively be designed such that the rate of release of the one or more biological agents from one or more regions on the medical device is the same or different. Non-limiting arrangements that can be used to control the release of one or more biological agent from the medical device include, but are not limited to, a) at least partially coating one or more biological agents with one or more polymers, b) at least partially incorporating and/or at least partially encapsulating one or more biological agents into and/or with one or more polymers, c) inserting one or more biological agents in pores, passageway, cavities, etc. in the medical device, d) inserting one or more biological agents in pores, passageway, cavities, etc. in the medical device and at least partially coating or covering such pores, passageway, cavities, etc. with one or more polymers and/or controlling the size and/or openings of the pores, cavities, passageways, etc., and/or e) incorporate one or more biological agents in the one or more polymers that at least partially form the medical device. As can be appreciated, other or additional arrangements can be used to control the release of one or more biological agent from the medical device. When the one or more biological agents are coated on the medical device, the one or more biological agents can be, but is not required to be, 1) directly coated on one or more surfaces of the medical device, 2) mixed with one or more coating polymers or other coating materials and then at least partially coated on one or more surfaces of the medical device, 3) at least partially coated on the surface of another coating material that has been at least partially coated on the medical device, and/or 4) at least partially encapsulated between a) a surface or region of the medical device and one or more other coating materials and/or b) two or more other coating materials. As can be appreciated, many other coating arrangements can be additionally or alternatively used. As can also be appreciated, one or more biological agents can be deposited on the top surface of the medical device to provide an initial uncontrolled burst effect of the one or more biological agents prior to 1) the control release of the one or more biological agents through one or more layers of polymer system and/or 2) the uncontrolled release of the one or more biological agents through one or more layers of polymer system. The one or more biological agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In yet another and/or alternative non-limiting aspect of the invention, the medical device can include a marker material that facilitates enabling the medical device to be properly positioned in a body passageway. The marker material, when used, is typically designed to be visible to electromagnetic waves (e.g., x-rays, microwaves, visible light, inferred waves, ultraviolet waves, etc.); sound waves (e.g., ultrasound waves, etc.); magnetic waves (e.g., MRI, etc.); and/or other types of electromagnetic waves (e.g., microwaves, visible light, inferred waves, ultraviolet waves, etc.). In one non-limiting embodiment, the marker material is visible to x-rays (i.e., radiopaque). The marker material can form all or a portion of the medical device and/or be coated on one or more portions of the medical device. The location of the marker material can be on one or multiple locations on the medical device. The size of the one or more regions that include the marker material can be the same or different.

In still yet another and/or alternative non-limiting aspect of the invention, the medical device can include biodegradable and/or bio-stable materials.

One non-limiting object of the present invention is the provision of a medical device that can be used to inhibit or prevent damage, injury and/or rupturing of diseased areas of a body passageway during the initial insertion of the medical device in a body passageway.

Another and/or alternative non-limiting object of the present invention is the provision of a medical device that can be used to at least partially encapsulate unstable and/or vulnerable plaque in a blood vessel.

Still another and/or alternative non-limiting object of the present invention is the provision of a medical device that can be used to at least partially repair and/or heal unstable and/or vulnerable plaque in a blood vessel.

Yet another and/or alternative non-limiting object of the present invention is the provision of a medical device that can be inserted and secured in a body passageway so as to limit or prevent damage, injury and/or rupture to a diseased area in the body passageway by the medical device prior to the medical device encapsulating the diseased area.

Still yet another and/or alternative non-limiting object of the present invention is the provision of a medical device that can apply one or more biological agents to a diseased area of a body passageway to facilitate in the repair and/or healing of the diseased area of a body passageway.

A further and/or alternative non-limiting object of the present invention is the provision of a medical device that is used to treat a unstable and/or vulnerable plaque in such a way that the treatment area is not disrupted, and the medical device is secured in the body passageway and the healing of the diseased area is completed in a timely fashion.

Still a further and/or alternative non-limiting object of the present invention is the provision of a medical device in the form of a stent. Yet a further and/or alternative non-limiting object of the present invention is the provision of a medical device that is at least be partially comprised of a biodegradable or biostable material. Still yet a further and/or alternative non-limiting object of the present invention is the provision of a medical device having a very thin wall thickness. Another and/or alternative non-limiting object of the present invention is the provision of a medical device that can be at least partially made of memory metal. Still another and/or alternative non-limiting object of the present invention is the provision of a medical device that can be at least partially formed of a metal with a biostable or biodegradable polymer coating. Yet another and/or alternative non-limiting object of the present invention is the provision of a medical device that can be at least partially balloon expandable. Still yet another and/or alternative non-limiting object of the present invention is the provision of a medical device that includes anchoring structures on at least one portion of the surface of the medical device. A further and/or alternative non-limiting object of the present invention is the provision of a medical device that includes a biological agent that is at least partially interdispersed on the medical device to propagate healing of a diseased and/or damaged area. Still a further and/or alternative non-limiting object of the present invention is the provision of a medical device that is deployed in a body passageway in such a manner so as not to disrupt or to minimize disruption of unstable and/or vulnerable plaque. Yet a further and/or alternative non-limiting object of the present invention is the provision of a medical device that includes VEGF, growth factors, stem cells and/or drugs.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 illustrates a section of blood vessel that includes an area of unstable and/or vulnerable plaque;

FIG. 2 illustrates a prior art stent expanded in a region of the unstable and/or vulnerable plaque and causing rupture the unstable and/or vulnerable plaque which results in a portion of the ruptured unstable and/or vulnerable plaque to travel downstream one or more the blood vessels;

FIG. 3 is side perspective view of the medical device in accordance with the present invention in a partially expanded state in the blood vessel;

FIG. 4 is side perspective view of the medical device in accordance with the present invention in a fully expanded state in the blood vessel;

FIG. 5 is side perspective view of a balloon designed to expand the end portions of the medical device;

FIG. 6 is side perspective view of another balloon designed to first expand the end portions of the medical device and then expand the intermediate portion of the medical device;

FIG. 7 is side perspective view of the medical device in accordance with the present invention in an expanded state and including a polymer coating on the body of the medical device;

FIG. 8 is side perspective view of the medical device in accordance with the present invention in a partially expanded state and including a polymer coating on the body of the medical device;

FIG. 9 is side perspective view of the medical device in accordance with the present invention in a partially expanded state and including a polymer film secured to a portion of the body of the medical device;

FIG. 10 is side perspective view of the medical device in accordance with the present invention in a partially expanded state and including a sheath on the body of the medical device; and,

FIG. 11 is side perspective view of the medical device in accordance with the present invention in a partially expanded state and including a sheath or sleeve on the interior portion of the body of the medical device.

### DETAILED DESCRIPTION OF THE PREFERRED EMBODIMENTS

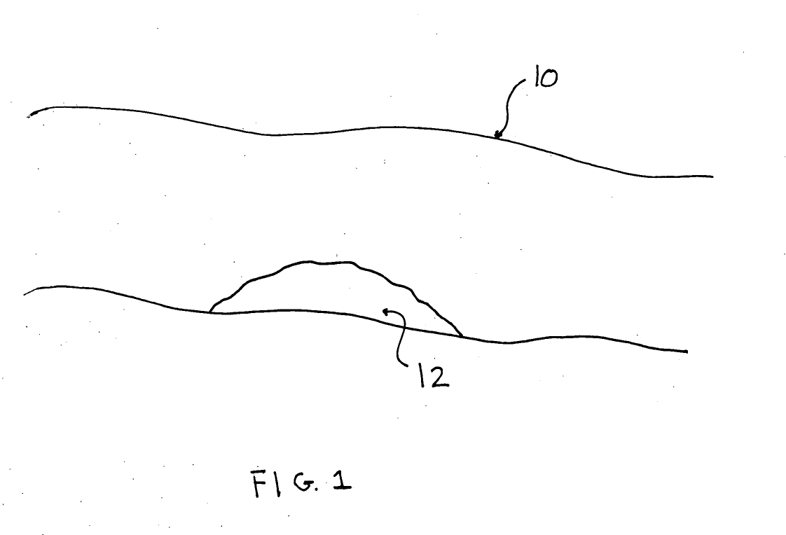

Referring now to the drawings wherein the showings are for the purpose of illustrating embodiments of the invention only and not for the purpose of limiting the same, FIG. 1 illustrates a blood vessel 10 that includes a diseased segment or area such as unstable and/or vulnerable plaque 12; however, it will be appreciated that the diseased area can be a disease other than or in addition to unstable and/or vulnerable plaque. Although the invention will be described with particular reference to blood vessels, it will be appreciated that the medical device of the present invention can be used in other types of body passageways.

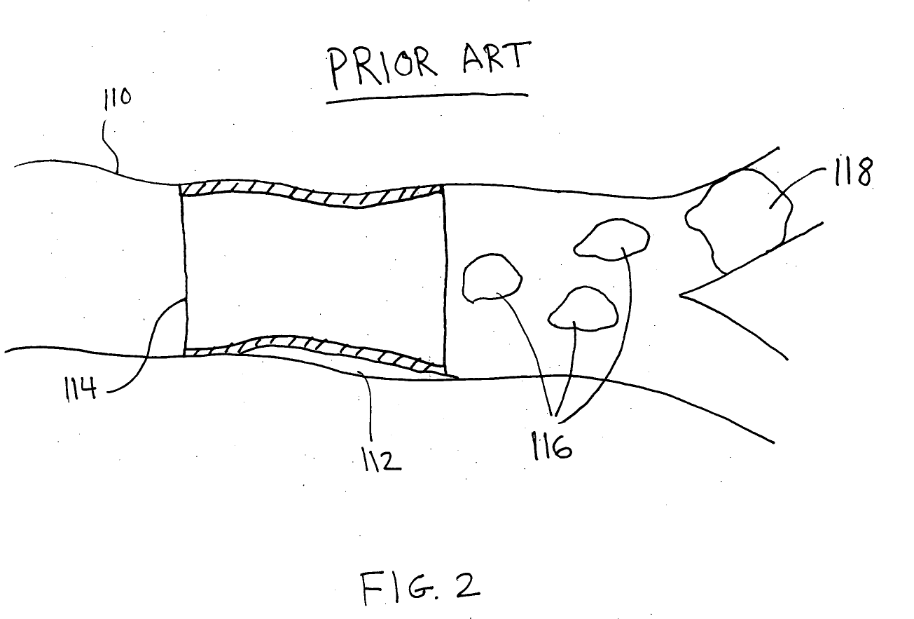

Referring again to FIG. 1, the unstable and/or vulnerable plaque 12 in the blood vessel 10 has a propensity to rupture when pressure is applied to the unstable and/or vulnerable plaque. As illustrated in FIG. 2, prior methods for treating blood vessels 110 that were obstructed by plaque 112 included the use of a stent 114 or angioplasty balloon which were expanded in the location of the plaque to cause the plaque to be compressed to the wall of the blood vessel when the stent or angioplasty balloon was expanded. The force directed on the plaque by the stent or an angioplasty balloon risked rupture of the plaque, which rupture could result in ruptured plaque 116 forming a blockage or clot 118 in the blood vessel downstream from the ruptured vulnerable as illustrated in FIG. 2. As illustrated in FIG. 2, portions of the released unstable and/or vulnerable plaque could penetrate through the mesh wall construction of prior art stents and then enter into the blood stream to be carried downstream. In addition, the length of the prior art stent was sometimes selected such that only a portion of the unstable and/or vulnerable plaque was contacted by the expanded stent and ruptured portions of the unstable and/or vulnerable plaque at the ends of the stent were allowed to freely travel into the blood vessel. These prior art stents also could cause tearing of the blood vessel wall resulting from a tear in the vulnerable plague. Such a tear could continue to grow and could result in restenosis about the expanded stent.

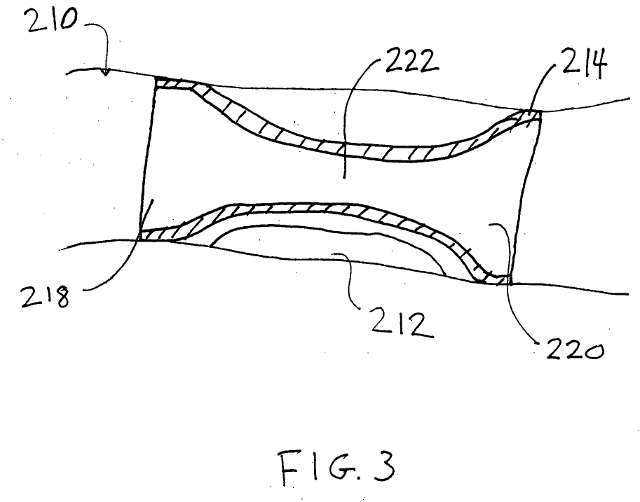

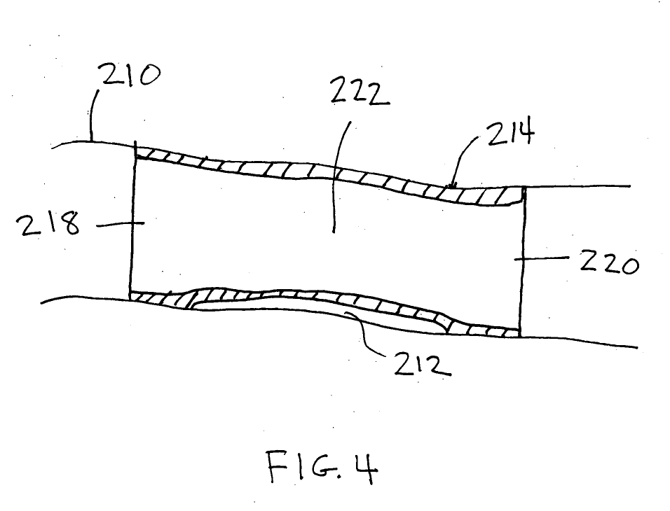

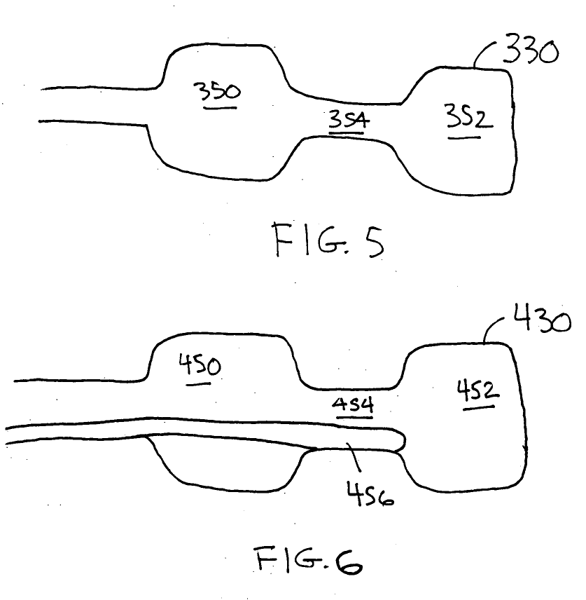

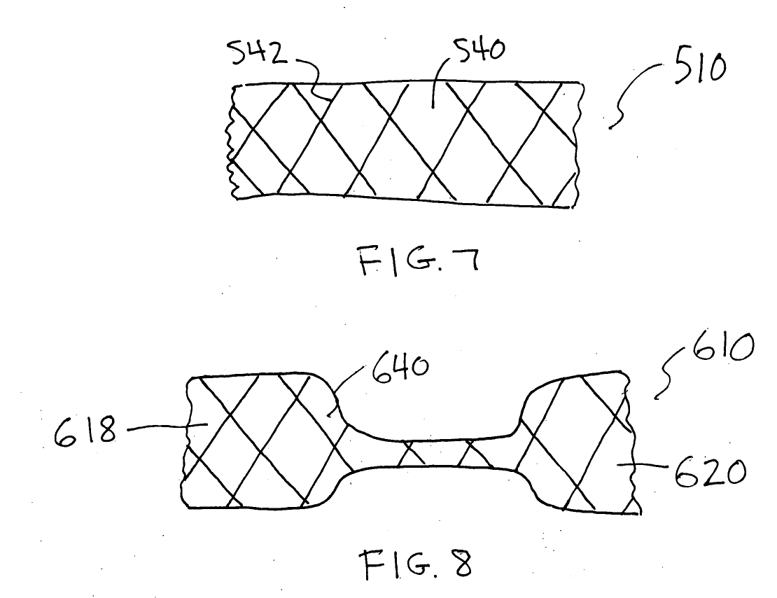

The medical device of the present invention is designed to address the problems that can occur when a blood vessel or other type of body passageway includes a diseased area. Referring now to FIG. 3, the stent of the present invention 214 is designed to be crimped on a delivery device such as, but not limited to, a catheter, not shown, so that the stent can be delivered to a treatment area in the blood vessel 210. The stent is selected to have a longitudinal length so that the end portions of the stent can be expanded and not contact the unstable and/or vulnerable plaque 212 as shown in FIG. 3. A balloon such as, but not limited to, balloons 330, 430 illustrated in FIGS. 5 and 6 can be used to partially expand the stent into a generally dog-bone shape as shown in FIG. 3. A discussion of the balloons in FIGS. 5 and 6 will be set forth in more detail below. As can be appreciated, other types of inflation devices can be used to inflate the stent (e.g., balloon catheter, etc.). The expansion of the end portions of the stent enable the stent to be secured or anchored in the blood vessel without damaging the unstable and/or vulnerable plaque.

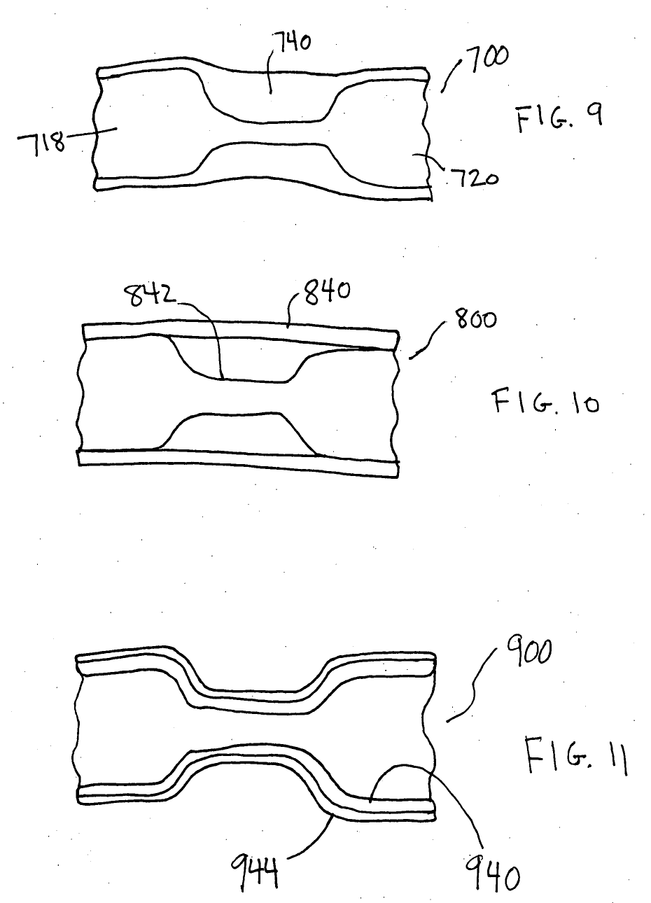

Once the stent has been positioned in the blood vessel 210 and the end portions 218, 220 have been expanded to anchor the stent in the blood vessel, the mid or intermediate portion 222 of the stent can be expanded as illustrated in FIG. 4. The stent of the present invention includes a flexible barrier layer that inhibits or prevents released portions of the unstable and/or vulnerable plaque from traveling downstream from the stent during and/or after the expansion of the intermediate portion of the stent. The flexible barrier can take many different forms. Three types of non-limiting flexible barriers are illustrated in FIGS. 7-11. The flexible barrier can be formed from a variety of materials. FIGS. 7-11 illustrate that the flexible barrier is a polymer material; however, this is not required. The flexible barrier is designed to encapsulate the unstable and/or vulnerable plaque between the flexible barrier and the inner wall of the blood vessel so that if or when the unstable and/or vulnerable plaque ruptures after the stent has been anchored in the blood vessel, the pieces of ruptured unstable and/or vulnerable plaque are retained in the region of the stent by the flexible barrier.

As illustrated in FIG. 3, the end portion of the stent is expanded and comes in contact with the inner wall of the blood vessel. The flexible barrier as illustrated in FIGS. 7-11 is thus moved into contact with or move into very close proximity to the inner wall of the blood vessel when the end portions of the stent are expanded; thereby forming end seal regions at the end portions of the stent. These two end seal regions form the encapsulated region for the unstable and/or vulnerable plaque. As such, if or when the unstable and/or vulnerable plaque ruptures, the ruptured portion of the unstable and/or vulnerable plaque is substantially inhibited or prevented from bypassing the two end sealed regions. The remainder of the flexible barrier is formed of a material which also substantially or fully prevents the ruptured portion of the unstable and/or vulnerable plaque from penetrating the flexible barrier. As such, the flexible barrier in combination with the inner wall of the blood vessel substantially or fully encapsulates the unstable and/or vulnerable plaque and substantially or fully prevents any ruptured plaque from escaping from the stented region of the blood vessel once the end portions of the stent have been expanded in the blood vessel.

Referring now to FIGS. 7 and 8, the flexible barrier is illustrated as a coated layer 540 on the body of the stent 510. The coated flexible barrier can be coated on the outer peripheral surface 542 and/or inner peripheral surface of the stent. FIG. 7 illustrates the coat flexible barrier when the stent has been expanded and FIG. 8 illustrates the coat flexible barrier 640 when the end portions 618, 620 of the stent 610 have been expanded for initially anchoring the stent in the blood vessel. The flexible barrier is typically formed of a flexible and stretchable material so that when one or more portions of the stent are expanded, the flexible barrier does not tear. The flexible barrier can be coated on the stent by variety of precesses such as, but not limited to, dip coating, spraying coating, roll coating, etc.

Referring now to FIG. 9, the flexible barrier 740 is illustrated as only being connected to the end portions 718, 720 of the stent 700. The flexible barrier can be preformed and then subsequently secured to the end portions of the stent by a variety of processes such as, but not limited to, adhesive, melt bond, clamp, etc. As can be appreciated, the flexible barrier can alternatively be integrally formed with the stent. Similar to the flexible barrier in FIGS. 7 and 8, the flexible barrier illustrated in FIG. 9 is typically formed of a flexible and stretchable material so that when one or more portions of the stent is expanded, the flexible barrier does not tear.

Referring now to FIGS. 10 and 11, the flexible barrier is in the form of a sheath or sleeve. The flexible barrier may or may not be secured to the body of the stent. If one or more portions of the flexible barrier are secured to the body of the stent, the flexible barrier can be secured to the stent by a variety of processes such as, but not limited to, adhesive, melt bond, clamp, etc. The flexible barrier 840 illustrated in FIG. 10 is shown to be fitted over the outer peripheral surface 842 of the stent 800. The flexible barrier 940 illustrated in FIG. 11 is shown to be inserted adjacent an inner peripheral 944 of the stent 900. The flexible barriers in FIGS. 10 and 11 are typically formed of a flexible and stretchable material so that when one or more portions of the stent is expanded, the flexible barrier does not tear.

A protective sheath, not shown, can be used to protect one or more portions of the flexible barrier from damage during the insertion of the stent into the treatment area; however, this is not required.

The body of the stent and/or the flexible barrier can include one or more biological agents to facilitate in the healing and/or repair of the unstable and/or vulnerable plaque; however, this is not required. When one or more biological agents are used, the one or more biological agent can be controllably or uncontrollable released.

In one non-limiting medical procedure, the stent is designed to be anchored in the blood vessel to substantially encapsulate the unstable and/or vulnerable plaque as illustrated in FIG. 3. The stent includes one or more biological agents that are selected to heal or repair the unstable and/or vulnerable plaque. After some passage of time (e.g., hours, days, weeks, months, etc.), the intermediate portion of the stent is expanded as illustrated in FIG. 4.

In another non-limiting medical procedure, the stent is designed to be anchored in the blood vessel to substantially encapsulate the unstable and/or vulnerable plaque as illustrated in FIG. 3. The intermediate portion of the stent is then expanded at a short time thereafter as illustrated in FIG. 4. The stent may or may not include one or more biological agents that are selected to heal or repair the unstable and/or vulnerable plaque.

The stent can include one or more markers on one or more portions of the stent (e.g., end portions of the stent body, flexible barrier, etc.) to facilitate in the positioning the stent in the blood vessel; however, this is not required.

Referring again to FIGS. 5 and 6, there are illustrated two novel balloon designs that can be used to expand the stent of the present invention. The balloon 330 illustrated in FIG. 5 includes two portions 350, 352 that expand to a greater cross-section area than the mid portion 354 of the balloon. The two larger expanding portions of the balloon are positioned under the two end portions of the stent so that when the balloon is expanded, the expanded balloon causes the stent to expand such as, but not limited to, the generally dog-bone shape as illustrated in FIG. 3. After the balloon is expanded, the balloon is deflated and removed from the stent. A standard balloon used to expand prior art stent, not shown, or other type of balloon can be subsequently be inserted in the interior region of the stent at some later time (e.g., few seconds, few minutes, few hours, few days, few months, etc.) and expanded to cause the intermediated portion of the stent to be expanded as illustrated in FIG. 4.

FIG. 6 illustrates a novel balloon design that can first expand the end portions 450, 452 of the stent and then expand the intermediate portion of the stent prior to removing the balloon from the stent. The balloon is illustrated as including a second inflation tubes 456. One inflation balloon 430 is used to expand the balloon in a form similar to the balloon illustrated in FIG. 5. The second inflation tube is used to expand the mid portion of the balloon so as to expand the intermediate portion of the stent after the end portions of the stent have been expanded. As can be appreciated, other balloon designs can be used to achieve similar results from the balloons illustrated in FIGS. 5 and 6.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

What is claimed is:

1. A medical device used to treat a unstable and/or vulnerable plaque in a blood vessel so as to minimize or prevent rupture of the unstable and/or vulnerable plaque at least until the unstable and/or vulnerable plaque is at least partially encapsulated by the medical device.

2. The medical device as defined in claim 1, wherein said medical device includes one or more portions designed to at least partially secure the medical device in the body passageway at a location that is proximal and/or distal to the unstable and/or vulnerable plaque.

3. The medical device as defined in claim 1, wherein said medical device at least partially repairs and/or heals the unstable and/or vulnerable plaque.

4. The medical device as defined in claim 1, wherein said medical device is a stent.

5. The medical device as defined in claim 1, wherein at least a portion of said medical device is comprised of a biodegradable or biostable material.

6. The medical device as defined in claim 1, wherein at least a portion of said medical device includes thin walls.

7. The medical device as defined in claim 1, wherein at least a portion of said medical device is made of memory metal.

8. The medical device as defined in claim 1, wherein at least a portion of said medical device is made of metal with a biostable or biodegradable polymer coating.

9. The medical device as defined in claim 1, wherein at least a portion of said medical device is balloon expandable.

10. The medical device as defined in claim 1, wherein at least a portion of said medical device includes anchoring structures on at least one portion of the surface of the medical device.

11. The medical device as defined in claim 1, wherein at least a portion of said medical device includes biological agent to propagate healing and/or repair of said unstable and/or vulnerable plaque.

12. The medical device as defined in claim 11, wherein said biological agent includes VEGF, growth factors, stem cells and/or drugs.

13. The medical device as defined in claim 1, wherein said medical device can be deployed in such a manner as not to disrupt the unstable and/or vulnerable plaque.

14. The medical device as defined in claim 1, wherein at least a portion of said medical device can be expanded by use of low pressures.

15. The medical device as defined in claim 1, wherein said medical device includes a flexible barrier, said flexible barrier including an amphiphilic block copolymer.
